(19) United States
(12) Patent Application Publication
(10) Pub. No.: US 2025/0312164 A1
(43) Pub. Date: Oct. 9, 2025

(54) GLENOID BASEPLATE AND IMPLANT ASSEMBLIES

(71) Applicant: HOWMEDICA OSTEONICS CORP., Mahwah, NJ (US)

(72) Inventors: Pierric DERANSART, Saint Martin d'uriage (FR); Pascal BOILEAU, Bloomington, MN (US); Gilles WALCH, Bloomington, MN (US); Charles L. PENNINGER, Bloomington, MN (US)

(73) Assignee: HOWMEDICA OSTEONICS CORP., Mahwah, NJ (US)

(21) Appl. No.: 19/243,090

(22) Filed: Jun. 19, 2025

Related U.S. Application Data
(63) Continuation of application No. 17/432,228, filed on Aug. 19, 2021.
(60) Provisional application No. 62/847,077, filed on May 13, 2019.

(51) Int. Cl. A61F 2/40; A61F 2/30

(52) U.S. Cl. CPC A61F 2/4081; A61F 2/30749; A61F 2002/30433; A61F 2002/30797; A61F 2002/30813

(57) ABSTRACT

A glenoid baseplate is provided that has a transverse body and an elongate body. The transverse body has a first side configured to engage scapula bone of a patient, a second side configured to face away from the first side, and a plurality of anchor apertures. The anchor apertures are formed between the first side and the second side. The transverse body also can have an arcuate or circular periphery that has an anterior portion configured to be oriented toward an anterior side of a scapula and a posterior portion that is configured to be oriented toward a posterior side of the scapula. The elongate body is disposed along a longitudinal axis between an end coupled with the first side of the transverse body. The longitudinal axis of the elongate body is off-set from the center of the circular periphery.

### INCORPORATION BY REFERENCE TO ANY PRIORITY APPLICATIONS

This application is a continuation filed under 37 C.F.R. § 1.53 claiming the benefit under 35 U.S.C. § 120 of any and all applications for which a foreign or domestic priority claim is identified in the Application Data Sheet as filed with the present application, including U.S. patent application Ser. No. 17/432,228, filed Aug. 19, 2021, International Patent Application No. PCT/US2020/031134, filed May 1, 2020, and U.S. Provisional Patent Application No. 62/847,077, filed May 13, 2019, and are hereby incorporated by reference in accordance with 37 C.F.R. §§ 1.57; 1.97; and 1.98 in their entireties.

### BACKGROUND OF THE INVENTION

### Field of the Invention

This application is directed to improved glenoid baseplate designs that are capable of being coupled to a scapula at a glenoid surface and that can have an anchor peg disposed medial of the glenoid surface and between anterior and posterior faces of the scapula.

### Description of the Related Art

Shoulder joint conditions can sometimes be resolved with shoulder arthroplasty. More and more, efforts are being focused on making total shoulder joint arthroplasty available to patients who would benefit from such treatment. In a total shoulder joint arthroplasty, the glenoid is typically reamed and a glenoid articular component is mounted to scapula following reaming. The articular component provides a smooth surface for movement of a humeral head or humeral articular component.

A glenoid baseplate can be used to support the glenoid articular component on the scapula. The glenoid baseplate can include an anchor peg on the medial side thereof that is configured to be inserted into scapular bone as part of securing the glenoid baseplate to the scapula.

### SUMMARY OF THE INVENTION

While glenoid baseplates are known, one challenge faced in implanting such baseplates is assuring that the anchor peg (or other medial projection) of the baseplate is consistently embedded in, e.g., fully enclosed within, the scapula bone and does not extend through a posterior or anterior wall of the scapula.

In some examples, methods are used to produce a glenoid baseplate that can include a elongate body, e.g., an anchor peg, that can be positioned on a transverse body of the baseplate in a position to extend into a deep part or even a deepest part of the scapula beneath the glenoid surface. The anchor peg position can be matched to the geometry beneath the glenoid (sometimes referred to as vault geometer) to provide a long or even a longest anchor peg that would fit in the portion of the vault selected for the anchor. In some cases the position is chosen such that the anchor peg does not perforate a wall of the scapula.

In one embodiment, a glenoid baseplate is provided that has a transverse body and an elongate body. The transverse body has a first side configured to engage scapula bone of a patient, a second side configured to face away from the first side, and a plurality of anchor apertures. The anchor apertures are formed between the first side and the second side. The transverse body also can have a circular periphery that extends between the first side and the second side. The circular periphery has an anterior portion configured to be oriented toward an anterior side of a scapula and a posterior portion that is configured to be oriented toward a posterior side of the scapula. The circular periphery can have a center. The elongate body is disposed along a longitudinal axis between a first end and a second end. The second end is coupled with the first side of the transverse body. The first end is disposed away from the second end. The longitudinal axis of the elongate body is off-set from the center of the circular periphery toward the anterior portion thereof.

Anchor apertures through the baseplate can be fixed relative to the baseplate such that a surgeon can advance a bone anchor through each anchor aperture without determining an angle or orientation of the anchor member during the time of surgery, eliminating a step of determining the angle or orientation.

Anchor apertures can be configured to enable the surgeon to select the angle of an anchor member relative to the baseplate or through the anchor aperture at the time of the surgery. Segmented threads can allow the angle of the anchor member through the anchor aperture to be selected at the time of surgery. Angling an internal member that is configured to tilt, rotate or swivel can allow for the selection of an orientation of the anchor member relative to the baseplate at the time of surgery.

In another embodiment, a glenoid baseplate is provided that includes a transverse plate and an anchor peg. The transverse plate has a medial side configured to engage scapula bone of a patient, a lateral side configured to face away from the medial side, a plurality of bone screw holes formed between the medial side and the lateral side, and a circular periphery. The circular periphery extends between the medial side and the lateral side. The circular periphery has an anterior portion configured to be oriented toward an anterior side of a scapula, a posterior portion configured to be oriented toward a posterior side of the scapula, and a center. The anchor peg is disposed along a longitudinal axis between a lateral end coupled with the medial side of the transverse plate and a medial end disposed away from the lateral end. The longitudinal axis of the anchor peg is off-set from the center of the circular periphery toward the anterior portion thereof.

In another embodiment, a method is performed or instructed in which a glenoid anchor is provided. The glenoid anchor has a transverse member and a projection. The transverse member has a medial side, a lateral side, an anterior periphery, and a posterior periphery. The projection extends from the medial side. The projection is located closer to the anterior periphery than to the posterior periphery. In the method, a blind hole is formed in a lateral portion of the scapula. The blind hole is formed along a trajectory that is off set from a center of an inferior portion of a glenoid. The blind hole has an opening at the glenoid and an enclosed end opposite the opening. The enclosed end is spaced apart from an anterior surface of the scapula. The enclosed end is spaced apart from a posterior surface of the scapula. The projection of the glenoid anchor is advanced into the blind hole such that the projection is enclosed within the scapula along the blind hole between the opening and the enclosed end.

In another embodiment, a method is performed in which image data responsive to a scan of a scapula of a patient is received. An anchor trajectory extending medially from a lateral surface of the scapula is identified from the image data. The anchor trajectory is at a selected position relative to, e.g., spaced apart from, an anterior surface of the scapula. The anchor trajectory can be at a selected position relative to, e.g., spaced apart from, a posterior surface of the scapula. The spacing of at least one of the anchor trajectory from the anterior surface or the anchor trajectory from the posterior surface is based on the imaging information. A glenoid anchor that has a transverse member and a projection is formed. The glenoid anchor has a medial side, a periphery bounding the medial side, a lateral side, an anterior portion, and a posterior portion. The projection extends from the medial side. The projection is disposed within the periphery at a location aligned with the identified anchor trajectory when the periphery is aligned with the curvature of the inferior portion of the glenoid rim.

In some cases, the periphery bounding the medial side corresponds to a curvature of portion of a glenoid rim of the patient. For instance, the periphery can correspond to an inferior portion of the glenoid rim of the patient. The periphery can correspond to a curvature of another portion of the glenoid if the inferior portion of the glenoid rim is subject to deformity. The periphery can be independent of the shape or curvature of the rim of the patient in some cases.

In some examples an approach is used in which some perforation of a wall of the scapula beneath the glenoid surface is desired, e.g., to provide for bi-cortical fixation of an anchor peg or other elongate member. This approach can be combined with an approach to select a location for the anchor peg or other elongate anchor member that extends into a relatively deep portion of the area under the glenoid (e.g., the vault).

### BRIEF DESCRIPTION OF THE DRAWINGS

Various embodiments are depicted in the accompanying drawings for illustrative purposes, and should in no way be interpreted as limiting the scope of the embodiments. Furthermore, various features of different disclosed embodiments can be combined to form additional embodiments, which are part of this disclosure.

FIG. 1 shows a model of a human humerus and a scapula with a glenoid, the glenoid having an articular assembly coupled thereto, the articular assembly including a glenoid baseplate with a medial end that projects through a posterior wall of the scapula, the humerus having a reverse implant assembly coupled thereto;

FIG. 1A is a schematic view of a glenoid and an imager that can be used to gather imaging information pertaining to the glenoid;

FIG. 1B is a flow chart of a method that can be performed with imaging information;

FIG. 1C is a schematic diagram of a portion of a scapula including a glenoid;

FIG. 1D is a flow chart of a method for providing for bi-cortical fixation while achieving sufficient anchor peg length;

FIG. 1E is a schematic diagram of a portion of a scapula including a glenoid;

FIG. 2 shows two views of a scapula with an articular assembly baseplate coupled thereto, a medial end of the baseplate projecting through posterior and anterior walls of scapulae;

FIG. 3 is a side view of one embodiment of a glenoid baseplate having an anteriorly offset anchor peg;

FIG. 3A is a lateral side view of the glenoid baseplate of FIG. 3;

FIG. 3B is a cross-section of the glenoid baseplate of FIG. 3;

FIG. 4 is side view of another embodiment of a glenoid baseplate having anteriorly offset anchor peg;

FIG. 4A is a lateral side view of the glenoid baseplate of FIG. 4;

FIG. 5 is a side view of another embodiment of a glenoid baseplate having anteriorly offset anchor peg and a lateral mounting projection with a relatively small diameter taper;

FIG. 5A is a lateral side view of the glenoid baseplate of FIG. 5;

FIG. 5B is a cross-section of the glenoid baseplate of FIG. 5;

FIG. 5C is side view of another embodiment of a glenoid baseplate having a centered anchor peg;

FIG. 6 is a side view of another embodiment of a glenoid baseplate having an anchor peg that is offset anteriorly to a position aligned with an aperture circumference;

FIG. 6A is a lateral side view of the glenoid baseplate of FIG. 6; and

FIGS. 7-7C illustrate various steps of a method of applying a glenoid baseplate to a scapula.

### DETAILED DESCRIPTION OF THE PREFERRED EMBODIMENT

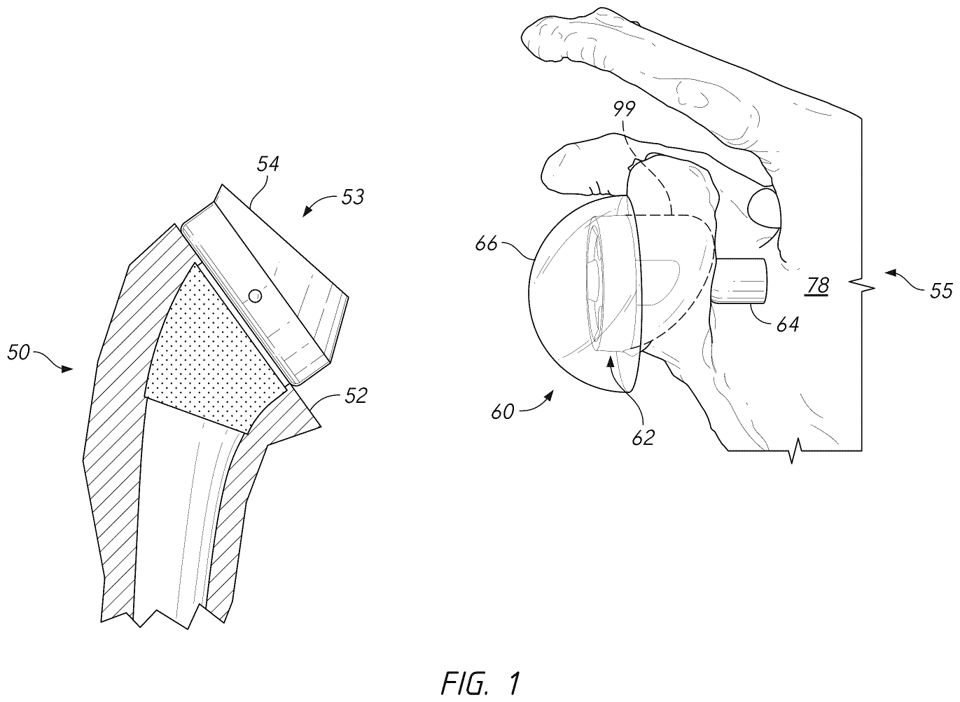

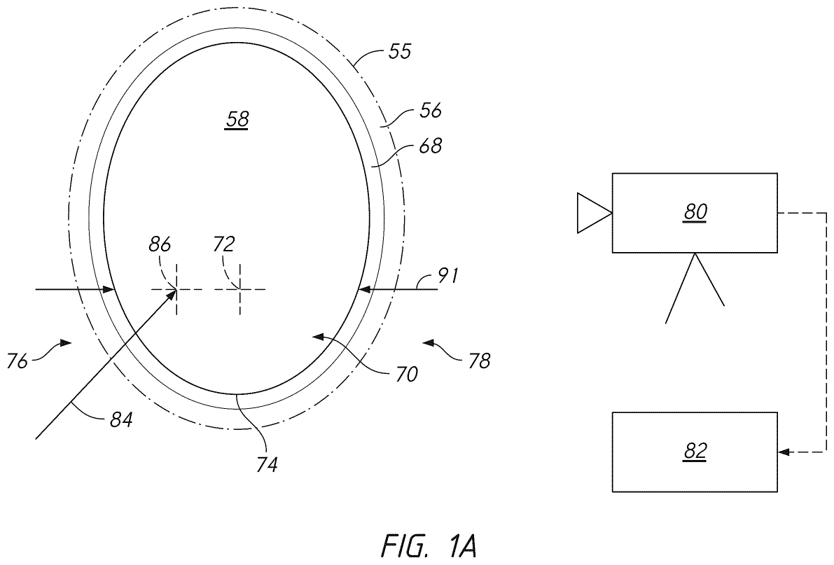

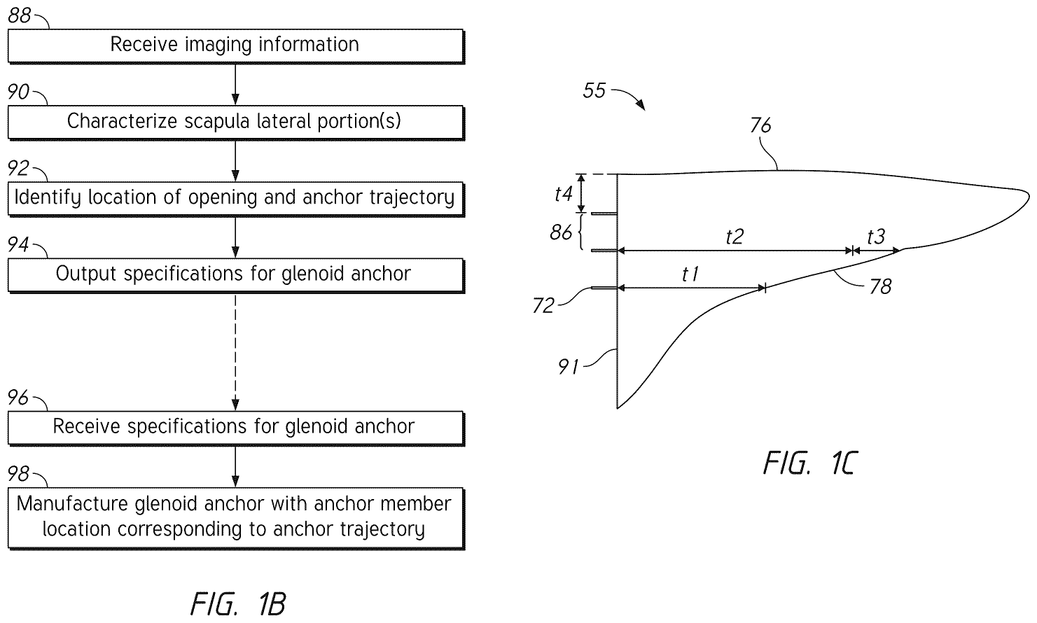

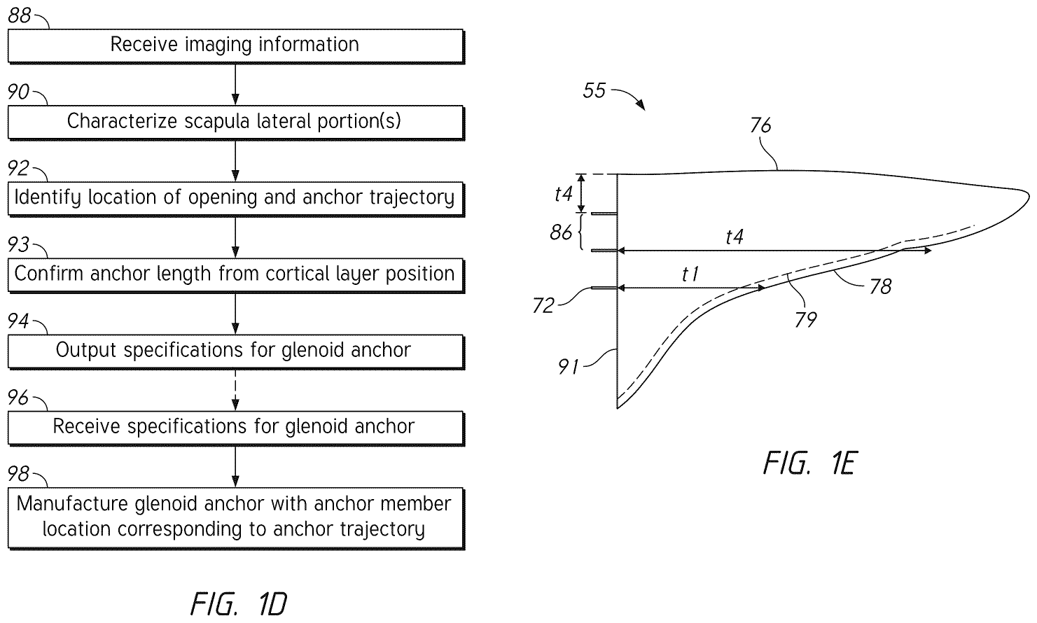

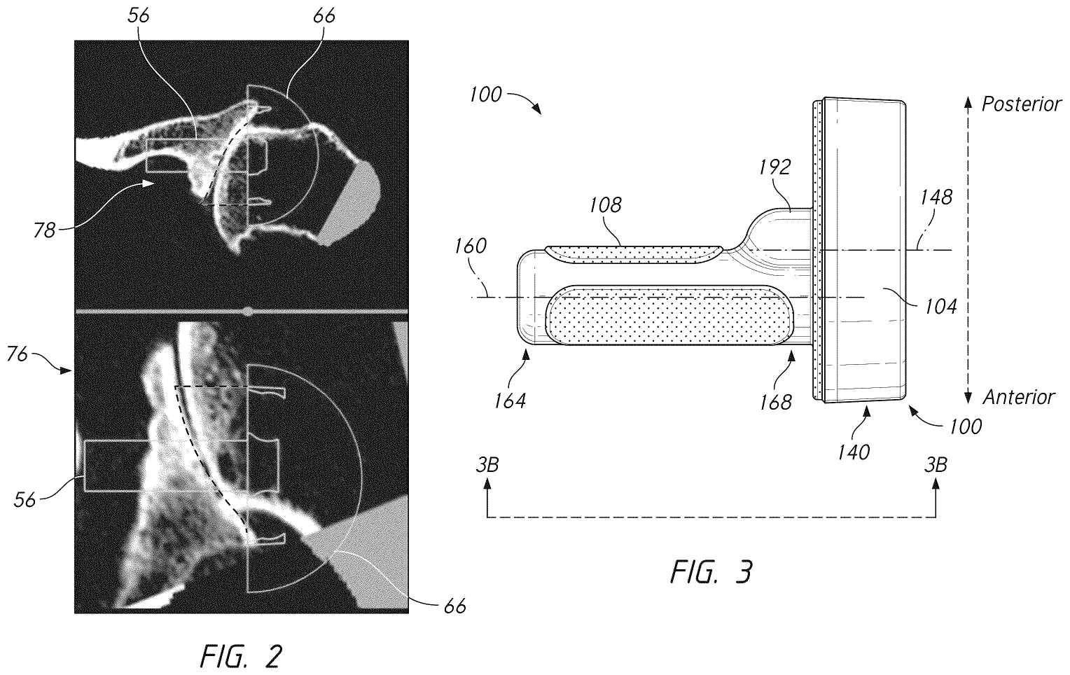

This application is directed to improving the success in providing sound connection between a glenoid assembly and a human scapula. These improvements are intended to allow for greater success in shoulder arthroplasty surgery. FIGS. 1 and 2 illustrate concerns that can arise in some shoulder procedure. FIG. 1 shows a schematic view of a humerus 50 and a scapula 55 of a shoulder having reverse shoulder implants disposed therein. The humerus 50 has a humeral resection 52. A humeral implant assembly 53 including a humeral anchor (shown beneath the humeral resection 52) and a reverse articular body 54. The reverse articular body 54 can be disposed above the humeral resection 52 or at least partially below the humeral resection 52 in some cases. The scapula 55 has a lateral surface 56 that includes a glenoid 58 (see FIG. 1A). The glenoid 58 is the portion of the scapula 55 on which the head of the humerus 50 normally articulates. Following total shoulder arthroplasty, this function is provided by an articular body 66 that is coupled to the scapula 55. For example, a glenoid assembly 60 can be provided that includes a glenoid baseplate 62 to support the articular body 66. The glenoid baseplate 62 can be coupled with the scapula 55. The glenoid baseplate 62 can have an anchor peg 64 configured to be advanced into the scapula 55. The glenoid baseplate 62 can have an end disposed on bone beneath the glenoid 58 if the glenoid 58 is reamed. In some embodiments described below the glenoid baseplate 62 can be placed on the glenoid 58 without or with minimal reaming.

In a sub-optimal case, the glenoid baseplate 62 is not properly placed on the scapula 55. FIGS. 1 and 2 show that the anchor peg 64 of the glenoid baseplate 62 can be placed into the scapula 55 in a sub-optimal manner in which a medial end of the anchor peg 64 pierces the posterior surface 78 of the scapula 55. The anchor peg 64 can be exposed outside the scapula 55 in that case. FIG. 2 shows that the medial end of the anchor peg 64 can pierce the anterior surface 76 or the posterior surface 78 of the scapula 55 These outcomes are sub-optimal for several reasons. The security of the connection between the anchor peg 64 and the bone of the scapula 55 is a function of the length over which there is direct contact between these structures. The direct contact provides opportunities for bony ingrowth, providing security. No such ingrowth will occur along a length that is completely exposed. Further, an exposed end could cause irritation to soft tissue around the scapula 55. Further, if the anchor peg 64 were to perforate the bone in an undesirable location the perforation could weaken the scapula and increase the risk of fracture.

FIG. 1A shows a schematic of a lateral side of a scapula 55. The glenoid 58 includes an articular surface separated from the rest of the glenoid 58 by a glenoid rim 68. A healthy shoulder joint will generally have within the glenoid rim 68 an elongate articular surface that has a generally circular inferior portion 70. More particularly the inferior portion 70 can be bounded by a circular segment of the glenoid rim 68. The circular portion of the glenoid rim 68 can be disposed about a center 72. More generally, the center 72 can be a central portion, e.g., a geometric center, of the inferior portion 70. The center 72 can be disposed on or along a infero-superior axis of the glenoid rim 68 that extends from the superior-most portion (located at the top of the graphic in FIG. 1A) of the glenoid rim 68 to the inferior-most portion (located at the bottom in FIG. 1A) of the glenoid rim 68. The center 72 may be located at a central portion, e.g., a mid-point, of a chord extending across the glenoid rim 68 at an infero-superior position disposed inferior of a geometric center of the entire glenoid 58 or glenoid rim 68. For example, the center 72 can be located about one-half to two-thirds of the distance from the inferior-most point of the glenoid rim 68 to the geometric enter of the entire glenoid 58 or glenoid rim 68.

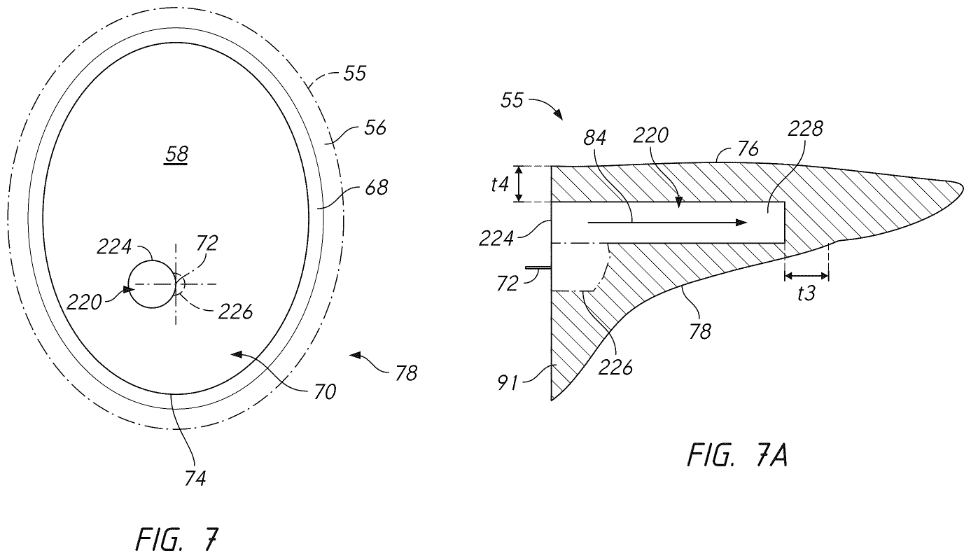

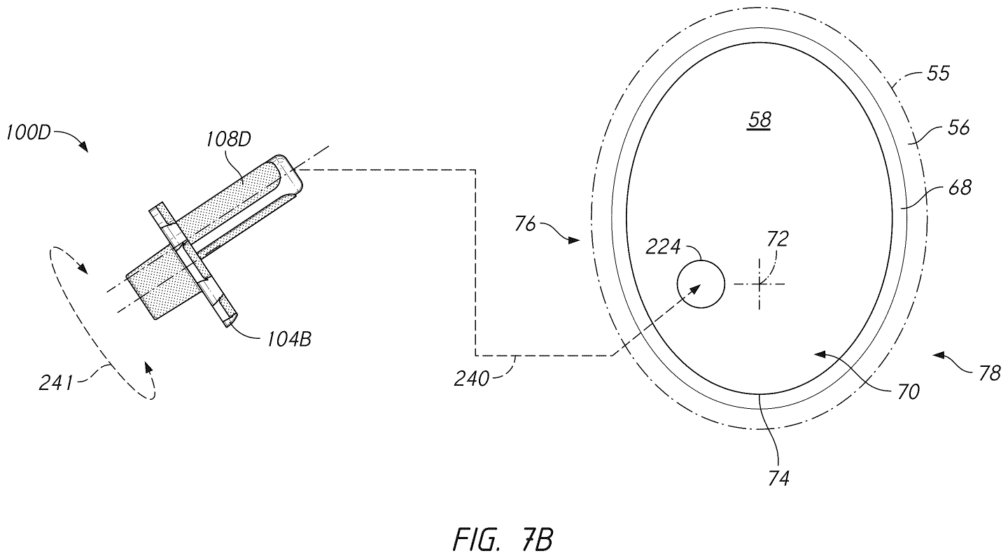

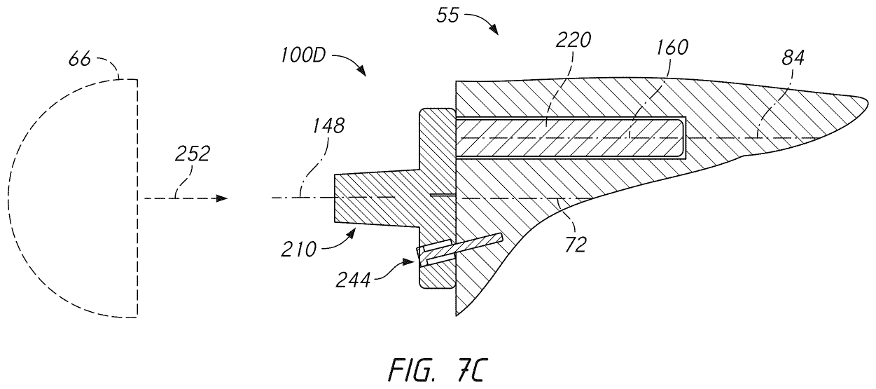

As will be discussed in greater detail below, an imager 80 can be used to scan the scapula 55 to gather imaging information. That information can be processed in an image processing system 82. The image processing system 82 can include a memory that can store imaging information corresponding to scanned data from the imager 80. The image processing system 82 can also include one or more hardware processors that can execute instructions. The image processing system 82 can process the imaging information to identify all the foregoing structures of the scapula 55. The imaging information can also be processed to locate an anchor trajectory 84 in a direction into the scapula 55 for placement of an anchor peg. The anchor trajectory 84 can be a direction from a blind hole 220 (discussed below in connection with FIG. 7A) in the lateral surface 56 of the scapula 55. The blind hole 220 can extend from an opening 224 (shown schematically in FIG. 1A as the opening 86) along the anchor trajectory 84. The anchor trajectory 84 can be offset from the center 72 of an inferior portion 74 of a glenoid 58. As shown in FIG. 1A the offset can be in the direction of the anterior surface 76. The opening 86 can be located between the center 72 and the anterior surface 76. The opening 86 can be located 10% of the distance from the center 72 to the anterior aspect of the glenoid rim 68 adjacent to the anterior surface 76 of the scapula 55. The opening 86 can be located 20% of the distance from the center 72 to the anterior aspect of the glenoid rim 68 adjacent to the anterior surface 76 of the scapula 55. The opening 86 can be located 30% of the distance from the center 72 to the anterior aspect of the glenoid rim 68 adjacent to the anterior surface 76 of the scapula 55. The opening 86 can be located 40% of the distance from the center 72 to the anterior aspect of the glenoid rim 68 adjacent to the anterior surface 76 of the scapula 55. The opening 86 can be located 50% of the distance from the center 72 to the anterior aspect of the glenoid rim 68 adjacent to the anterior surface 76 of the scapula 55. The opening 86 can be located 60% of the distance from the center 72 to the anterior aspect of the glenoid rim 68 adjacent to the anterior surface 76 of the scapula 55. In some cases, the opening 86 is located in a direction other than toward the anterior surface 76, e.g., closer to the posterior surface 78 of the scapula 55 by any of these or other percentages. The opening 86 could be in other directions as well, e.g., inferior, superior, or some direction between any of anterior, posterior, inferior, or superior depending on the needs of the patient.

The image processing system 82 can be configured to process imaging information in any suitable manner. FIG. 1B shows one method that can be performed at least in part by the image processing system 82. In a step 88 a process can receive the imaging information. For example, the imager 80 can be connected by a network to a computer having a processor configured to receive the imaging information. The network can include an Internet connection, a wireless or wired connection within the same facility where the imager 80 is located. In some applications, a data file including the imaging information can be physically transported to an image processing computer. In some applications, the imager 80 is directly connected to a computer with a processor configured to process the imaging information.

Thereafter in a step 90 a lateral portion or surface 56 of a scapula 55 can be characterized. The characterization of the lateral portion can include segmentation to create a virtual model of all or a portion of the scapula 55. The step 90 can include forming a virtual model of all or a portion of the humerus 50. The step 90 can include forming a virtual model of all or a portion of the glenoid 58. A virtual model formed in step 90 can include a model of the glenoid rim 68. The virtual model formed in step 90 can include a model of an inferior portion 70 of the glenoid rim 68. In step 90, the center 72 of the inferior portion 70 can be identified in the virtual mode. The glenoid 58 can be characterized to locate the center 72, e.g., by obtaining a radius of curvature of the inferior portion 70. The center 72 can be identified as the center for a radius of curvature of the inferior portion 70.

The step 90 can include characterizing a lateral surface 56 of the scapula 55. The scapula 55 can be disposed in the immediate vicinity of the glenoid 58, e.g., lateral facing bone disposed around the glenoid 58. In some cases, the scapula 55 is further characterized medially of the lateral surface 56, e.g., along an anterior surface 76 and/or along a posterior surface 78 of the scapula 55. The step 90 can include determining the thickness of the scapula 55 between the anterior surface 76 and the posterior surface 78 at one or more locations of the glenoid 58. For example, thicknesses can be determined along the line 91 as shown in FIG. 1A and 1C. FIG. 1C provides a simplified illustration of the line 91 as a straight line. In most examples of the glenoid 58 the line 91 follows the curvature of the glenoid 58, e.g., having a concavity facing laterally. The line 91 can extend from an anterior side of the glenoid rim 68 to a posterior side of the glenoid rim 68. A thickness t1 can be determined as a distance extending medially from the center 72. The distance can be measured perpendicular to a tangent line of the glenoid 58 at the center 72.

The step 90 can also include determining a thickness t2 at a location spaced apart from the center 72 if the thickness t1 is not sufficient to fully contain the anchor peg 64 of a glenoid baseplate 62. The location of the opening 86 can be determined as a location where the sum of a thickness t2 and a thickness t3 exceed a threshold. In one method, it is preferred to provide the thickness t2 from the end of a blind hole 220 that can be formed to retain the anchor peg 64 to the exterior wall of the posterior surface 78. For example, the thickness t3 can be at least large enough to leave the cortical wall at the posterior surface 78 intact. In one technique, the thickness t3 is measured from a portion of the blind hole 220 that is closest to the nearest wall of the scapula 55. For example if the posterior wall of the scapula 55 is sloped anteriorly in the vicinity of the end of the blind hole 220, the measurement for t3 would be from the posterior aspect of the blind hole 220 (rather than from the center of the blind hole) to the posterior wall.

FIG. 1C shows that in some cases, the scapula 55 can have a shape that allows the opening 86 to be placed over a range of positions. A distance from the location of the measurements thickness t2 and thickness t3 to the location of a measurement of a thickness t4 from the anterior surface 76 of the scapula 55 provides a range of possible locations for the opening 86. Upon identification of the range of positions for the opening 86, selection of the location of the opening 86 can be based on other factors such as bone quality below the glenoid 58 along the range of positions show in FIG. 1C. In some methods, the presence and location of any deformity can be considered. For example, the presence and location of a deformity can impact the ability of peripheral screws to be secured to the bone. So, the location of the opening 86 can be adjusted or selected to provide the best overall anchorage including that provided by peripheral screws.

The image processing system 82 can perform the step 92 in which the location of the opening 86 and the anchor trajectory 84 are determined. The image processing system 82 can determine the dimensions t2 and t3 as a part of determining the location of the opening 86 and the anchor trajectory 84. For example, a hardware processor in the image processing system 82 can execute code implementing a method that determines the thicknesses t2 and t3 for a given location offset from the center 72. At a location disposed an incremental distance anteriorly from the center 72, the image processing system 82 can determine the dimension t3. The thickness t3 can provide an anatomic reference distance, such as the thickness of the cortical wall adjacent to the anterior or posterior wall of the scapula beneath the glenoid (see FIG. 1C). The thickness t2 can be determined by the image processing system 82 from glenoid surface and the thickness t3 (see FIG. 1C). If the thicknesses t2 and t3 are sufficient for a given patient, the anchor trajectory 84 as well as the location for the opening 86 can be established. If either thickness t2 or t3 is not sufficient, a further increment from the center 72 can be evaluated by the image processing system 82. The condition at the bone corresponding to this further increment, e.g., the thicknesses t2 and t3, can be evaluated by the image processing system 82 to determine if the thicknesses t2 and t3 are sufficient. In some embodiments the image processing system 82 performs additional steps of the method of FIG. 1B, e.g., to generate a configuration for a glenoid baseplate 62. The configuration of the glenoid baseplate 62 can include an amount of offset between a center of a proximal or distal (or lateral or medial) portion of the glenoid baseplate 62 and the location of the center of the anchor peg 64. The direction along which the anchor peg 64 extends can be generally perpendicular to a lateral or medial surface of the glenoid baseplate 62 in some embodiments. In some methods, the image processing system 82 concludes the step 92 upon determining the location of the opening 86 and the corresponding position of the anchor peg 64 as well as the anchor trajectory 84 within the scapula 55 and the corresponding configuration (e.g., orientation and length) of the anchor peg 64. The opening 86 may advantageously be determined to be located anterior of the center 72, posterior of the center 72, inferior of the center 72, superior of the center 72, or any combination of anterior, posterior, inferior and superior to the center 72 as needed based on the analysis in step 90.

FIGS. 1 and 1C shows that the direction of the opening 86 relative to the center 72 is toward the anterior surface 76. The opening 86 could be located between the center 72 and the posterior surface 78, as shown in FIG. 2. A range of positions for the opening 86 between the center 72 and the posterior surface 78 also can be provided if the posterior surface 78 extends more generally medially-laterally and the anterior surface 76 is more curved toward the posterior surface (as in the lower image of FIG. 2).

In a step 94, a specification or configuration for a glenoid baseplate 62 can be output. The output can be in the form of drawings. The output can be computer code to be used by a rapid manufacturing facility. The output in step 94 can be sent directly or indirectly to multiple recipients, including a review recipient, a manufacturing recipient, a physician customer and/or a patient customer.

In step 96 the configuration or specifications output in step 94 can be received by a manufacturing facility. The configuration or specification can be received by other parties in the step 96. The step 96 can involve a 3D printer of any sort to receive instructions output in the step 94. The instructions can be received and can be implemented by the 3D printer forming the glenoid baseplate 62 in a step 98. The step 98 generate the glenoid baseplate 62 by forming the glenoid baseplate 62 and thereafter putting the glenoid baseplate 62 through appropriate finishing processes. The step 98 can include transferring the glenoid baseplate 62 to the surgeon immediately upon concluding the method of FIG. 1B or subsequently.

FIGS. 1D-1E illustrate further variations of methods and apparatuses herein. The method illustrated in FIG. 1D is similar to that of FIG. 1B except that there is an additional step 93 in which the length of an elongate body, e.g., an anchor peg, which may be described as an anchor length, is confirmed. An anchor length can correspond to a thickness t4 between a glenoid surface. In one technique the determination of the thickness t4 can include using the image processing system 82 to identify a cortical boundary 79, e.g., a location where generally cancellous bone transitions to generally cortical bone. The thickness t4 can be required to be at least the distance from the glenoid surface to the cortical boundary 79. The thickness t4 can be required to be greater than this distance, such that the result is that a glenoid baseplate formed with an elongate body defined by the method of FIG. 1D reaches and in some cases extends through the scapula wall to provide bi-cortical fixation. The rest of the method of FIG. 1D can be the same as the method of FIG. 1B.

A patent application filed under attorney docket number 126-033USP1 on May 13, 2019 listing inventors Pierric Deransart and Vincent Simoes and bearing to title Patient-Matched Orthopedic Implant is hereby incorporate by reference herein in its entirety for further disclosure of various methods related to configuring and making various embodiments of glenoid baseplates and other orthopedic implants in a patient specific or patient-matched manner and for further disclosure of such glenoid baseplates and orthopedic implants as well as for all other purposes.

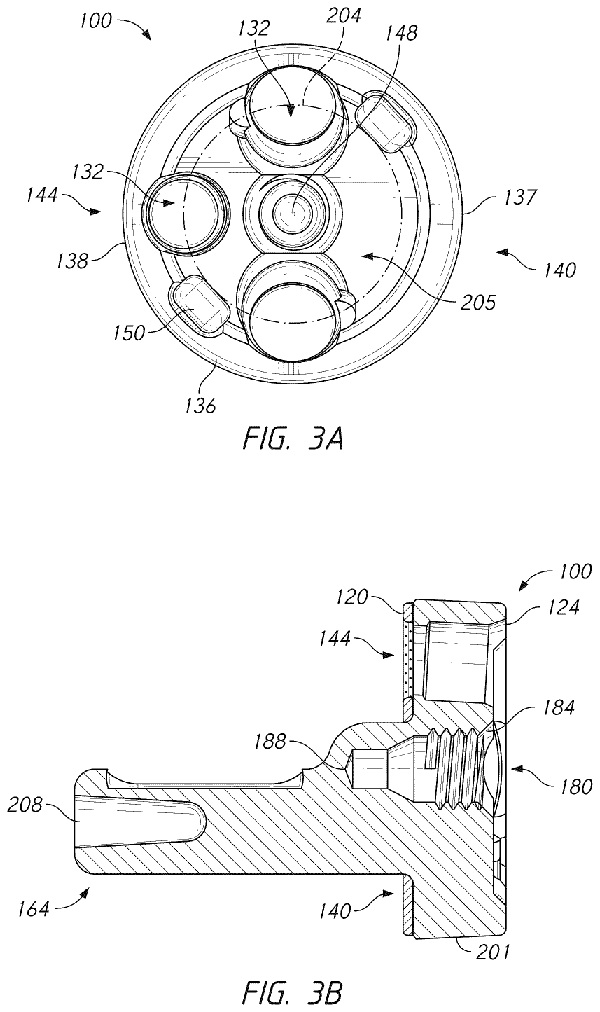

FIGS. 3-3B shows an example glenoid baseplate 100 according to one embodiment. The glenoid baseplate 100 can be used to secure a glenoid articular member to a glenoid as discussed below. The glenoid baseplate 100 is an example of an implementation of the glenoid baseplate 62 discussed above.

The glenoid baseplate 100 includes a transverse body 104 and an elongate body 108. The elongate body 108 can be configured as an anchor peg, e.g., similar to the anchor peg 64. The glenoid baseplate 100 includes a first side 120. The first side 120 can be a medial side configured to engage scapula bone of a patient. The glenoid baseplate 100 includes a second side 124. The second side 124 can be a lateral side configured to face away from the medial side 120. The second side 124 can face away from the scapula 55 when the first side 120 is secured against the scapula 55.

FIG. 3A shows that the glenoid baseplate 100 can include plurality of anchor apertures 132, which can be a plurality of bone screw holes, formed between the medial side 120 and the lateral side 124. The anchor apertures 132 can be evenly distributed about the transverse body in some embodiments. In some embodiments, there is an uneven distribution of the anchor apertures 132 about the second side 124. The anchor apertures 132 can be fixed holes or fixed passages through the baseplate 100. The anchor apertures can be formed as holes or passages defined in part by or through internal members that are disposed within recesses in the baseplate 100. In some embodiments, the recesses and/or apertures 132 in the baseplate 100 can include a non-threaded and/or threaded surface. In various examples one or more of two or more apertures 132 can be threaded and another one or more of the two or more apertures 132 can be non-threaded. In an example, two threaded apertures 132 can be disposed through an augmented portion of the first or medial side 120, e.g., an angled surface thereof, and one or more, e.g., two, non-threaded holes can be disposed adjacent to the threaded holes. The augmented portion can be a pre-defined augment appropriate for the patient or can be a patient-specific augmented portion that is designed specifically for the patient based on imaging of the scapula and/or glenoid region of the patient. In some embodiments, the internal member(s) can include a semi-spherical outer surface and can be disposed in a recess that is semi-spherical, in order to permit movement, e.g., rotation, tilting and/or swiveling, of the internal member with respect to the baseplate 100. The internal member(s) allow an orientation of bone anchors relative to the baseplate 100 to be selected at the time of a surgery. Further details of internal members that can tilt, rotate or swivel are described in U.S. Pat. No. 9,629,725B2, which is hereby incorporated herein by reference.

The glenoid baseplate 100 can have a circular periphery 136 that can extend between the medial side 120 and the lateral side 124. The circular periphery 136 can be tapered, e.g., larger toward the first side 120 than toward the second side 124. The circular periphery 136 can correspond to a curvature of an inferior portion 74 of a glenoid rim 68 of the scapula 55. The elongate body 108 can be disposed within the circular periphery 136 at a location to be aligned with the anchor trajectory 84 when the periphery is aligned with the curvature of the inferior portion 74 of the glenoid rim 68. The elongate body 108 can be aligned with the opening 86 by rotationally orienting the glenoid baseplate 100, e.g., by orienting an indicia of directionality as directed. A notation such as “SUP” can be marked on the transverse body 104 to be aligned with the superior direction. Other indicia of directionality can be provided in other embodiments.

FIG. 3A shows that the circular periphery 136 of the glenoid baseplate 100 can have a center 148 with a feature for securing an articular body to the glenoid baseplate 100 as discussed further below. The circular periphery 136 can have an anterior periphery 137 comprising a portion of the circular periphery 136. The circular periphery 136 can have a posterior periphery 138. The posterior periphery 138 comprises a posterior portion of the circular periphery 136. The elongate body 108 can be configured as a projection that extends from the medial side 120. The projection 108 can be located closer to the anterior periphery 137 than to the posterior periphery 138.

The glenoid baseplate 100 can include an anterior portion 140 configured to be oriented toward an anterior side of a scapula 55. The glenoid baseplate 100 can include a posterior portion 144 configured to be oriented toward a posterior side of the scapula 55. The anterior portion 140 and the posterior portion 144 can disposed around, e.g., can surround the center 148.

FIG. 3 shows how the transverse body 104 and the elongate body 108 can be secured or coupled to each other. The elongate body 108 can extend along a longitudinal axis 160. The elongate body 108 can have a first end 164 and a second end 168. The second end 168 can be coupled with the first side 120 (e.g., with the medial side) of the transverse body 104. The first end 164 can be disposed away from the second end 168 (e.g., away from the lateral end). The first end 164 can be a medial end 164 of the glenoid baseplate 100. The longitudinal axis 160 of the anchor peg 108 is off-set from the center 148 of the circular periphery 136 toward the anterior portion 140. The center 148 is illustrated by a dashed line in FIG. 3. FIGS. 3A and 3B show that a recess is formed at the center 148. The recess allows a connection feature (e.g., a screw) to be used to secure an articular body (such as the articular body 66) to the glenoid baseplate 100. The configuration of the glenoid baseplate 100 allows the connection between the articular body 66 and the glenoid baseplate 100 to be along the center 148 while allowing the location of the elongate body 108 to be spaced apart from the center 148, e.g., according to the method of FIG. 1B.

FIGS. 3A and 3B show that the glenoid baseplate 100 can have a threaded hole 180 to facilitate connection of an articular body (e.g., the articular body 66) to the glenoid baseplate 100. The threaded hole 180 preferably is configured as a blind hole with an open end 184. The blind hole configuration can include an enclosed end 188. The enclosed end 188 greatly increases the strength of the glenoid baseplate 100 compared to where the threaded hole 180 would open up on the medial side of the structure. The threaded hole 180 can be formed within a fastener body 192. The fastener body 192 can be configured as a transverse extension of the elongate body 108 as seen most prominently in FIG. 3. The fastener body 192 can have a width that is less than the diameter of the elongate body 108 at the first end 164 thereof. The fastener body 192 can extend by a distance that is less than the medial-lateral thickness of the transverse body 104. The fastener body 192 can include a first end 194 and a second end 196. The second end 196 can be coupled with the first side 120 of the transverse body 104. The first end 194 can be disposed away from the transverse body 104. The fastener body 192 enables the fastener to extend by at least a small distance beyond the transverse body 104. This configuration allows the transverse body 104 to be relatively or completely flat on the second side 124.

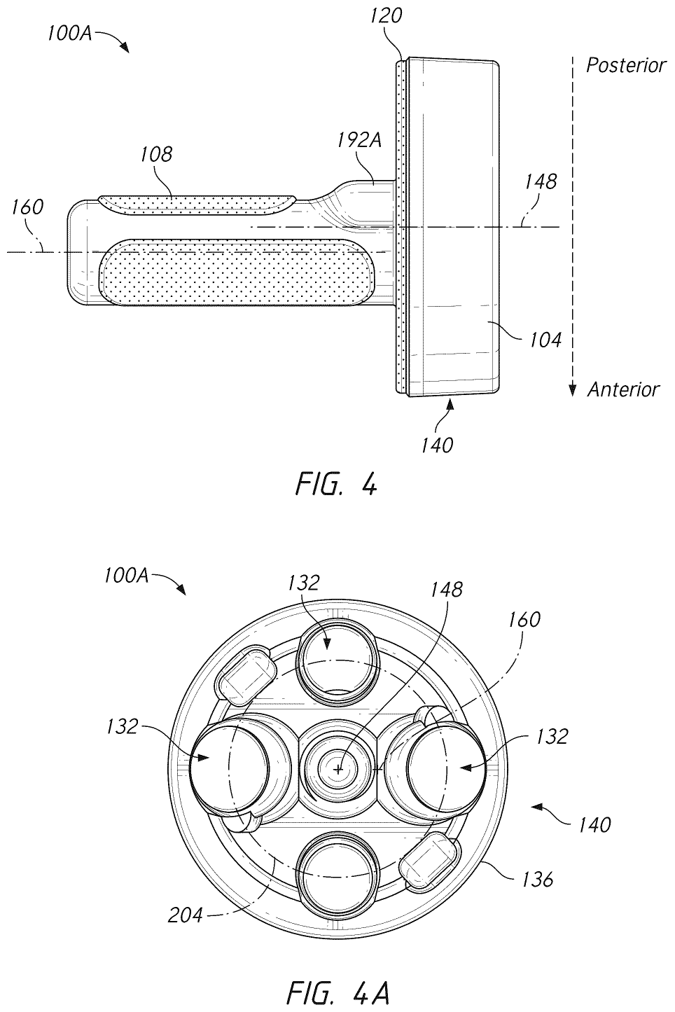

The circular periphery 136 of the transverse body 104 comprises a tapered profile 201 configured to mate with a tapered recess of the articular body 66. The second side 124 of the glenoid baseplate 100 can have an aperture circumference 204 wherein the anchor apertures 132 are evenly distributed about the lateral face of the transverse body 104. FIG. 4A shows an embodiment where all of the anchor apertures 132 are evenly spaced about a lateral face of the transverse body 104. FIG. 3A shows that an anterior portion of an aperture circumference 204 opposite the elongate body being formed without an aperture, e.g., providing a continuous area 205. The continuous area 205 can include a solid surface at a regular spacing from adjacent anchor apertures 132. The presence of a continuous area 205 can arise due to the shifting of the position of the elongate body 108 into the area of the anchor apertures 132. Because the elongate body 108 is solid there is not an opportunity to direct a bone screw through that area. Therefore, the continuous area 205 can be provided. The continuous area 205 can be a solid or unperforated area.

In some embodiments, a medial interface 208 is provided in the first end 164 of the elongate body 108. The medial interface 208 can include a blind hole that is centered on the distal face of the elongate body 108. The medial interface 208 could be used to secure patient bone matter or graft matter. A blind hole allows a pin or peg to be inserted into the elongate body 108 during handling or processing of the glenoid baseplate 100. During such holding the glenoid baseplate 100 can be further processed to provide appropriate smoothed surfaces. The handling interface 208 is optional and can be replaced with a solid surface at the second end 168 of the elongate body 108.

Handling of the glenoid baseplate 100 can be facilitated by providing a tooling interface 150 on the transverse body 104, e.g. on the second side 124 thereof. As discussed above, the second side 124 can be a lateral side of the glenoid baseplate 100. The second side 124 can be accessible by a tool that can extend through the skin to the surgeon in use when the tool has been used to place the glenoid baseplate 100 through the skin toward the glenoid. The tooling interface 150 can include two or more opposed slots, openings or blind holes in the second side 124 of the transverse body 104. The slots, openings or blind holes can be angled toward each other such that the ends thereof are closer to each other than are the ends of the slots, openings or blind holes at the surface of the second side 124. As shown, the tooling interface 150 can be disposed just outside of the aperture circumference 204 between two circumferentially adjacent anchor apertures 132.

FIGS. 4 and 4A show a glenoid baseplate 100A that is similar to the glenoid baseplate 100 except as described differently below. The description of the glenoid baseplate 100 supplements the description of the glenoid baseplate 100A. Any features of the glenoid baseplate 100A that are compatible with the glenoid baseplate 100 also supplement to the glenoid baseplate 100. The glenoid baseplate 100A includes the transverse body 104 and the elongate body 108 as discussed above. The elongate body 108 extends along the longitudinal axis 160. The transverse body 104 can have a circular periphery 136 with a center 148. An offset between the center 148 and the elongate body 108 can be provided. The offset can be in the anterior direction, as shown in FIG. 4. Depending on the nature of the scapula to which the glenoid baseplate 100A is to be applied the offset between the elongate body 108 and the center 148 can be in another direction, e.g., in the posterior direction. The offset between the elongate body 108 and the center 148 can be in other directions as well, e.g., inferior, superior, or some direction between any of anterior, posterior, inferior, or superior depending on the needs of the patient.

The glenoid baseplate 100 can have a fastener body 192A that is disposed alongside the elongate body 108. The fastener body 192A can be similar to the fastener body 192. For instance, the fastener body 192A can have an enclosed end 188 and an open end 184. The fastener body 192 can have a threaded hole 180 that extends from the open end 184 to the enclosed end 188. The fastener body 192A can be provided to allow at least a portion of a fastener coupled to the glenoid baseplate 100A to be disposed below a bone surface to which the first side 120 is applied. The fastener coupled to the fastener body 192A can extend out of the open end 184 and into an articular body to additionally secure the reverse articular body 66 to the glenoid baseplate 100A. Accordingly, a portion of a fastener can be disposed in the reverse articular body 66, a portion in the thickness of the transverse body 104 and a portion in the fastener body 192A.

The fastener body 192A extends away from a side surface of the elongate body 108 by a lesser extent than doe the fastener body 192 in the glenoid baseplate 100. Placement of the glenoid baseplate 100A requires less preparation of the bone of the glenoid 58 to accommodate the lesser volume of the fastener body 192A compared to the fastener body 192. Also, the location of the elongate body 108 closer to the fastener body 192A allows the periphery of the elongate body 108 to be disposed inward of the anchor apertures 132 in the transverse body 104. FIG. 4A shows that the configuration allows the aperture circumference 204 to include anchor apertures 132 that are evenly distributed entirely around the transverse body 104. For example, the transverse body 104 can have anchor apertures 132 located between the position of the elongate body 108, e.g., of the longitudinal axis 160, and the anterior portion 140 of the circular periphery 136. The glenoid baseplate 100A advantageously enables the elongate body 108 to be lodged completely in bone medial of the first side 120 of the transverse body 104 and also allows anchors to be disposed through the anchor apertures 132 at an even distribution providing even securement to the scapula.

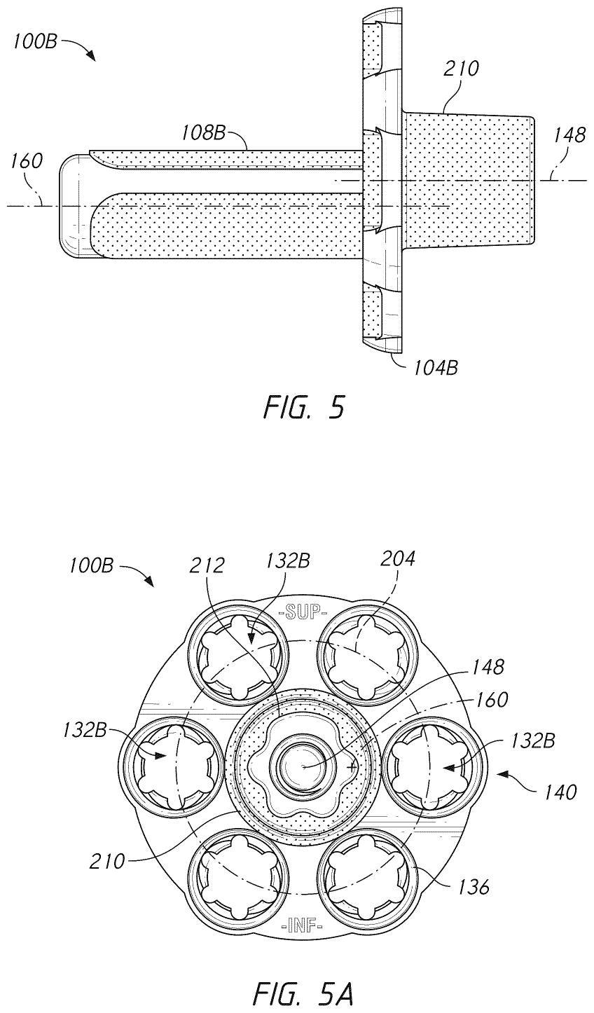

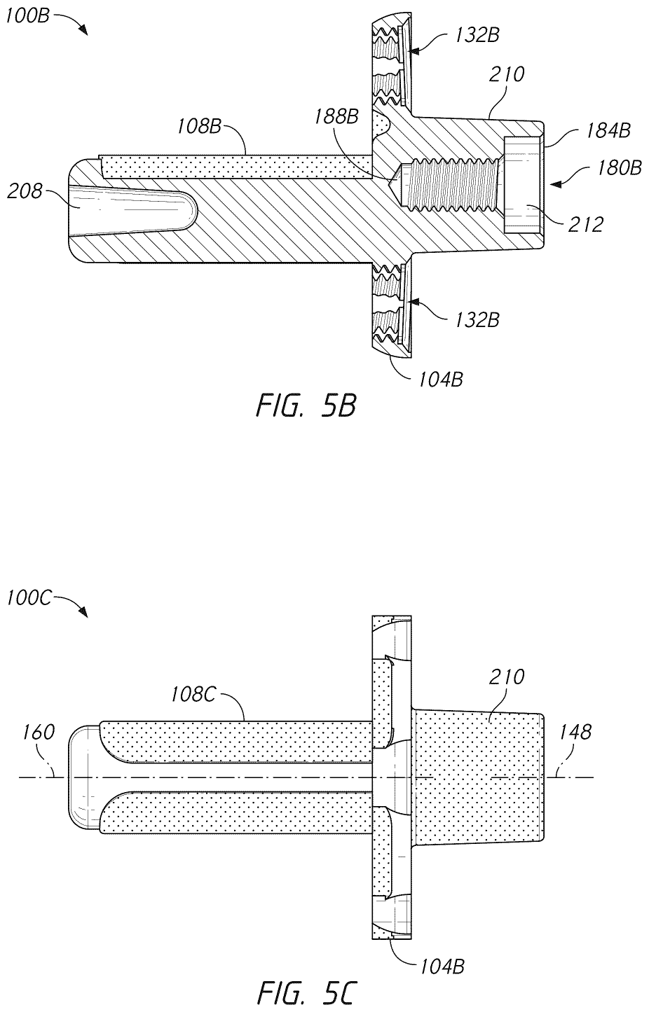

FIGS. 5-5B show a glenoid baseplate 100B that is similar to the glenoid baseplate 100 and the glenoid baseplate 100A except as described differently below. The descriptions of the glenoid baseplate 100 and the glenoid baseplate 100A supplement the description of the glenoid baseplate 100B. Any description of features of the glenoid baseplate 100B that are compatible with the glenoid baseplate 100 or the glenoid baseplate 100A also supplements that of the glenoid baseplate 100 and/or the glenoid baseplate 100A.

The glenoid baseplate 100B includes an elongate body 108B and a transverse body 104B. The elongate body 108B is disposed along a longitudinal axis 160. The transverse body 104B is symmetrical about a center 148, which is illustrated as a center axis 148. The glenoid baseplate 100B includes a coupling projection 210 disposed a side of the transverse body 104B opposite the elongate body 108B. The coupling projection 210 can be symmetrical about the center 148. The coupling projection 210 can have a tapered outer periphery. The coupling projection 210 can have an outer periphery that reduces in diameter along the length thereof from the transverse body 104B to a free end of the coupling projection 210. The coupling projection 210 can include a threaded hole 180B formed therein to engaged with the articular body 66. The threaded hole 180B can be disposed between an open end 184B and an enclosed end 188B within the coupling projection 210. The enclosed end 188B can be disposed within the thickness of the transverse body 104B. The enclosed end 188B can be disposed between the elongate body 108B and the coupling projection 210. A tooling interface 212 can be disposed in the threaded hole 180B. The tooling interface 212 can extend from the open end 184B toward the enclosed end 188B.

The tooling interface 212 can be rotationally asymmetric to contribute to visualizing or providing the correct orientation of the glenoid baseplate 100B relative to the scapula. For example, an arrangement of concavities can be provided about the tooling interface 212. An enlarged concavity can be provided at a superior portion of the tooling interface 212. The glenoid baseplate 100B also can be provided with visual indicia of orientation, e.g., labeled “SUP” for superior and/or “INF” for inferior on the lateral side of the transverse body 104B. In other embodiments, the transverse body 104B can be labeled “ANT” for anterior or “POS” for posterior. Other indicia could be used for indicating one or more of these orientations.

The transverse body 104B can include anchor apertures 132B that are similar to the anchor apertures 132 and can also include threads for connecting the transverse body 104B to bone anchor, e.g., screws, that can be advanced therethrough. The threads can be segmented to allow the threads to be secured to bone anchors in a fixed orientation, which orientation can be selected at the time of a surgery.

One advantage of the configuration of the glenoid baseplate 100B is that the elongate body 108B can have a cylindrical shape with a circular profile from a first end located at a first, bone-facing or medial side of the transverse body 104B to a second (medial) end of the elongate body 108B. The exterior surface of the elongate body 108B is free of a structure similar to the fastener body 192 or fastener body 192A because the faster advanced into the threaded hole 180B does not extend beyond the first, bone-facing or medial side of the transverse body 104B. This allows the preparation of the scapula bone of a patient to be simpler in that a circular hole can be formed in the recess allowing the glenoid baseplate 100B to be more easily inserted into the bone. The presence of the fastener body 192 or the fastener body 192A may require either supplemental preparation of the bone or compaction of the bone during impaction of the glenoid baseplate 100 or the glenoid baseplate 100A if the baseplate is prepared without a patient specific medial surface configured to be placed on the glenoid. In cases where the medial surface is made patient specific the fastener body 192 or the fastener body 192A may be located in an expanse of the baseplate that is augmented for the patient on the medial side of the baseplates and may be located laterally of the glenoid surface when applied. Thus, no additional bone preparation would be necessary for the fastener body 192 or the fastener body 192A.

The coupling projection 210 can provide a connection to the articular body 66 that is similar to the connection provided by the tapered profile 201 of the glenoid baseplate 100 or the glenoid baseplate 100A. The coupling projection 210 has a tapered profile that has larger dimensions toward the transverse body 104B than adjacent to the free end of the coupling projection 210. The largest dimension of the coupling projection 210 is smaller than the diameter of the side of the transverse body 104B to which the coupling projection 210 is coupled. Thus, the coupling projection 210 can be smaller than the transverse body 104B. The coupling projection 210 can have an aspect ratio that is greater than 1:5 (height to diameter of the projection 210). In various examples, the coupling projection 210 has an aspect ratio that is greater than 1:4, greater than 1:3, greater than 1:2, e.g., about 1:1. In contrast, the transverse body 104, the periphery of which provides the tapered profile 201 for mating to the articular body 66, can have an aspect ratio that is smaller than 1:1 (diameter to height of the body 104). The transverse body 104 can have an aspect ratio smaller than 1:2, smaller than 1:3, smaller than 1:4. In some example, the transverse body 104 has an aspect ratio that is between 1:2 and 1:7, e.g., about 1:5. The insertion and alignment of the articular body 66 with the smaller coupling projection 210 can be easier than the insertion and alignment of the articular body 66 over the generally larger tapered profile 201. The alignment of the body 66 to the smaller diameter projection 210 is easier in part because of the higher aspect ratio (length over width) of this feature. That is, the projection 210 is narrower and longer and thus has a much higher aspect ratio than the tapered profile 201, which is shorter and wider. The higher aspect ratio allows the articular body 66 to be placed over the free end of the coupling projection 210 and as it is advanced toward the transverse body 104B, the projection 210 guides the body 66 into the proper alignment prior to full connection. In the case of the tapered profile 201, the smaller aspect ratio provides less alignment confirmation by way of the structure of these features before the components are to be fully connected. With the presence of soft tissue and limited visibility the alignment is just more difficult. However, both interfaces provide excellent connection between the articular body 66 and the corresponding glenoid baseplate.

FIG. 5C shows a glenoid baseplate 100C that is similar to the glenoid baseplate 100B except as described differently below. The glenoid baseplate 100C has similarity to some feature of the glenoid baseplate 100 and the glenoid baseplate 100A as well. The descriptions of the glenoid baseplates 100, 100A, 100B supplement the description of the glenoid baseplate 100C. Any description of features of the glenoid baseplate 100C that are compatible with the glenoid baseplates 100, 100A, 100B also supplements that of the glenoid baseplates 100, 100A, and/or the glenoid baseplate 100B.

FIG. 5C shows that the glenoid baseplate 100C has an elongate body 108C that is disposed on a medial side of the transverse body 104B. The transverse body 104B is disposed on the center 148 as in the glenoid baseplate 100B. The elongate body 108C is disposed along a longitudinal axis 160. The longitudinal axis 160 is aligned with, e.g., intersects or is colinear with the center 148 of the transverse body 104B. As such, the glenoid baseplate 100C provide a configuration in which both the coupling projection 210 and the elongate body 108C are symmetrical about a common axis. The glenoid baseplate 100C is suitable for patients with more symmetrical anterior surface 76 and posterior surface 78 and/or patients with thicker a scapula. These patients can receive the full length of the elongate body 108C along a direction perpendicular to a plane of the transverse body 104B extending away from the first side 120 of the transverse body 104B when the anchor trajectory 84 is aligned with the center 72 of the glenoid 58.

The configuration of FIG. 5C can have the coupling projection 210 and the tooling interface 212. The glenoid baseplate 100 can be formed with a symmetrical configuration similar to the glenoid baseplate 100C.

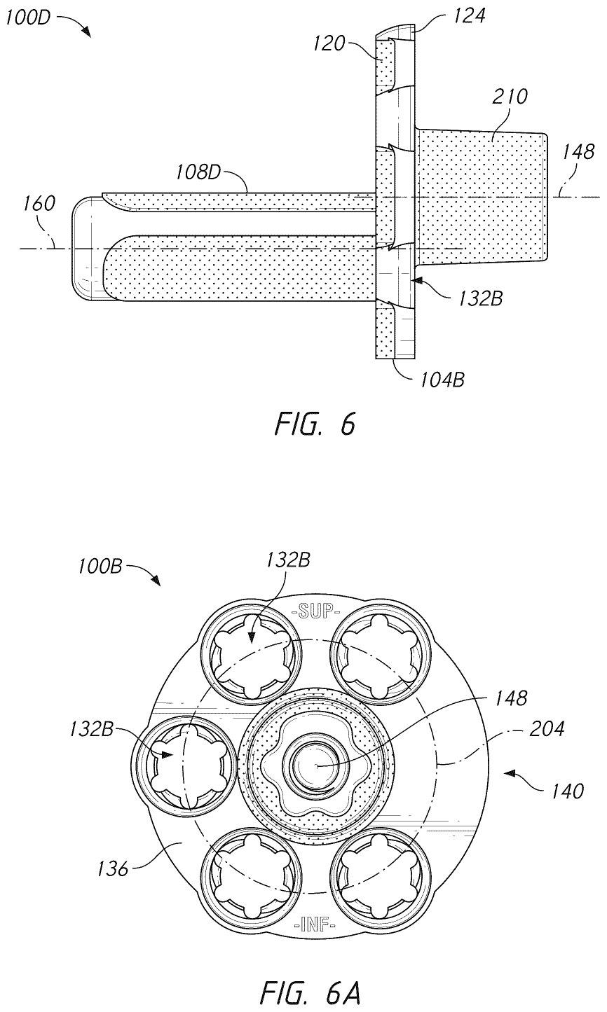

FIG. 6 shows a glenoid baseplate 100D that is similar to the glenoid baseplates 100, 100B except as described differently below. The glenoid baseplate 100D has similarity to some feature of the glenoid baseplates 100A, 100C as well. The descriptions of the glenoid baseplates 100, 100A, 100B, 100C supplement the description of the glenoid baseplate 100D. Any descriptions of features of the glenoid baseplate 100D that are compatible with the glenoid baseplates 100, 100A, 100B, 100C also supplements that of the glenoid baseplate 100 and/or the glenoid baseplate 100, 100A, 100B, 100C.

The glenoid baseplate 100D has a transverse body 104B and an elongate body 108D. The elongate body 108D is similar to the elongate body 108B. The elongate body 108D extends from a first side 120 of the transverse body 104B to a free end disposed away from the first side 120. The elongate body 108D extends along a longitudinal axis 160. The longitudinal axis 160 can be offset from a center 148 of a coupling projection 210 disposed on a side of the transverse body 104B opposite to the elongate body 108D. The offset between the center 148 and the longitudinal axis 160 can be in an anterior direction. Such an offset will accommodate a patient where more bone stock is available toward the anterior surface 76 than toward the posterior surface 78 of the scapula 55.

The offset between the longitudinal axis 160 and the center 148 of the glenoid baseplate 100D or the glenoid baseplate 100 can be about 6 mm in one embodiment. The offset between the longitudinal axis 160 and the center 148 of the glenoid baseplate 100D or the glenoid baseplate 100 can be about 5 mm in one embodiment. The offset between the longitudinal axis 160 and the center 148 of the glenoid baseplate 100D or the glenoid baseplate 100 can be about 4 mm in one embodiment. The offset between the longitudinal axis 160 and the center 148 of the glenoid baseplate 100B or the glenoid baseplate 100A can be about 3 mm in one embodiment. The offset between the longitudinal axis 160 and the center 148 of the glenoid baseplate 100B or the glenoid baseplate 100A can be about 2 mm in one embodiment. The offset between the longitudinal axis 160 and the center 148 of the glenoid baseplate 100B or the glenoid baseplate 100A can be about 1 mm in one embodiment. The offset between the longitudinal axis 160 and the center 148 of the glenoid baseplate 100B or the glenoid baseplate 100A can be about 0.5 mm in one embodiment.

FIG. 6A shows that for some amounts of offset the glenoid baseplate 100D omits one or more anchor apertures 132. As in the glenoid baseplate 100, the aperture circumference 204 provide that anchor apertures 132B are evenly distributed about the second side 124 of the transverse body 104B. The anchor apertures 132B can be spaced apart by even increments. In one or more of the increment positions the transverse body 104B is made solid with no anchor apertures 132B formed therein. The anchor apertures 132B is omitted because the glenoid baseplate 100D has the elongate body 108D disposed at least partially within the area of the anchor apertures 132B that is omitted. Because the elongate body 108D is positioned at this location a bone anchor, such as a bone screw, cannot be placed though this area.

### Shoulder Implant Configured From Surgical Planning

The various glenoid baseplates can be better understood with reference to FIGS. 1A-1C, discussed above. The glenoid baseplate 100 can advantageously be formed following step 88 in which imaging information of a scapula 55 is obtained from the imager 80. The imager 80 can be a CT scanner, X-ray, MRI or other similar imaging modality or from a tracer that can be obtain bone shape by direct contact with the bone or with tissue over the bone. The scapula 55 can be characterized in the step 90. This can involve any suitable technique employed by the image processing system 82 for creating a virtual model of the scapula 55, e.g., by segmenting the imaging information to provide a 3D model that can be stored in memory and/or displayed in a graphical user interface. This information can include identifying a center 72 of an inferior portion 70 of the glenoid 58. The center 72 can be identified as a mid-point of a chord of the inferior portion 74 of the glenoid rim 68 of the scapula 55. The chord can be located a distance equal to the radius from the inferior-most point of the inferior portion 74 of the glenoid rim 68. The chord can be located at an inferior-superior position along the glenoid rim 68 between an anterior portion thereof adjacent to the anterior surface 76 of the scapula 55 and a posterior portion thereof adjacent to the posterior surface 78 of the scapula 55. The inferior superior position can be that position where the distance across the glenoid 58 is equal to two times the radius of curvature of the inferior portion 74 of the glenoid rim 68. The glenoid baseplate 100 can be configured such that the transverse body 104 has a round periphery with a radius equal to the radius of curvature of the inferior portion 74 of the glenoid rim 68. The center 148 of the transverse body 104 can be located at the center 72 as determined in the step 90.

The position of the elongate body 108 can be determined in the step 92. The step involves finding an appropriate position for preparing the opening 86 in the surface of the glenoid 58 for receiving the elongate body 108. As noted above, the position may be positioned adjacent to the center 72. The opening 86 may advantageously be formed anterior of the center 72, posterior of the center 72, inferior of the center 72, superior of the center 72, or any combination of anterior, posterior, inferior and superior to the center 72 as needed based on the analysis in step 90. The position of the opening 86 may be selected by finding a location along the line 91 where any combination of the depth, strength, thickness, bone density and bone strength is sufficient to accommodate a minimum length of the elongate body 108. The position of the opening 86 can be one in which the bone will support formation of a blind hole in the scapula 55 extending from the opening 86 to an enclosed end where the entire length of the elongate body 108 can be accommodated in the blind hole. With reference to FIG. 1C the elongate body 108 can have a length that equates to the thickness t2. The positon can be anywhere between the two horizontal lines bounding reference number 86.

The step 94 can include specifying the diameter of the transverse body 104 and the position of elongate body 108. The step 94 can also include specifying the length of the elongate body 108. For patients with larger volume of bone in the scapula 55 the elongate body 108 can be made longer than the minimum length if such additional length is deemed to provide a clinical advantage, e.g., better implants security. The step 94 can also output a baseplate with a threaded hole that extends below the bone surface (as in FIGS. 3-4A) or that is located laterally of the bone when applied to the patient (as in FIGS. 5-6A).

The step 98 can include forming the baseplate through a suitable process such as an additive manufacturing process, e.g., three dimensional printing. Examples of three dimensional printing include direct metal laser sintering (DMLS), fused deposition modeling (FDM), fused filament fabrication (FFF), and electron beam melting (EBM). Any one or a combination of these or other additive manufacturing processes can be used in the step 98. In these processes a three dimensional object is formed by sequentially forming individual layers of the object on top of previously formed individual layers. These processes can closely control the gross dimensions of the object and also can form complex features and shapes such as contours. In certain embodiments, the step 98 can be used to form the first side 120 of any of the glenoid baseplates disclosed herein as complementary surface that can mate with specific anatomy of a specific patient, e.g., with a convex surface that mates with the concave surfaces of the glenoid 58 as show in dash-dot line 99 of FIG. 1.

As discussed further above, the scapula 55 has a thickness between the anterior surface 76 and the posterior surface 78 the scapula can be small and can rapidly and/or unpredictably decrease beneath the glenoid 58. Thickness change beneath the glenoid 58 can be due to many factors including the anterior and posterior surfaces of the scapula being irregular or inconsistent from patient to patient. The foregoing method and apparatus provide for consistently sound anchorage of the projection of the elongate bodies of the glenoid baseplates on the medial side of the baseplate within the wall of the scapula. The applicants of this application have found that for many patient this results in an offset medial post where the post is offset to an anterior zone of the baseplate. These configurations can lead to higher likelihood of clinical success.

### Surgical Methods For Baseplates Configured Based on Patient Scapula Anatomy

The foregoing disclosure provides for various advantageous surgical methods. FIGS. 7-7C illustrate some of these methods. FIG. 7 shows that a blind hole 220 can be formed in a lateral surface 56 of the scapula 55, e.g., within the surface of the glenoid 58. The blind hole can be formed at location of the opening 86 identified in the method of FIGS. 1A-1C. The blind hole can extend from an opening 224 corresponding to the opening 86 to an enclosed end 228 opposite the opening 224. The enclosed end 228 can be at a depth and at an anterior-posterior position that is spaced apart from the anterior surface 76 and the posterior surface 78 of the scapula 55. The blind hole can extend along an axis that corresponds to the anchor trajectory 84 determined in the method of FIGS. 1A-1C. FIGS. 7 and 7A show that the blind hole 220 is off set from the center 72 of the inferior portion 70 of a glenoid 58.

FIG. 7 shows that for some of the baseplate configurations, the blind hole 220 may be enlarged, e.g., providing an expanded opening 226. The expanded opening 226 can be a portion that extends from the blind hole 220 toward the center 72. For example, the glenoid baseplate 100A includes the fastener body 192A that is aligned with the center 148. The center 148 is to be aligned with the center 72 when the glenoid baseplate 100A is applied. Accordingly the surface of the glenoid 58 may be prepared to enlarge the blind hole 220 include the expanded opening 226 to accommodate the fastener body 192A. The expanded opening 226 is disposed at, over or around the center 72 such that a screw for connecting the articular body 66 to the glenoid baseplate 100A can be centered on the transverse body 104. Forming the expanded opening 226 may be accomplished by a surgical drill, by a small reamer, by a tamp, or in the process of placing a trial implant in the glenoid 58. Or the glenoid baseplate 100A could be advanced into the blind hole 220 and the expanded opening 226 can be formed by compression of the bone as the glenoid baseplate 100A is impacted into the bone.

FIG. 7C shows application of the glenoid baseplate 100D to the glenoid 58. The elongate body 108D can be advanced as indicated by an arrow 240 toward the glenoid 58 such that the elongate body 108D is at the opening 224 to the blind hole 220. The elongate body 108D can be advanced into the blind hole 220 until the elongate body 108D is adjacent to or contacts the enclosed end 228. The elongate body 108D can be advanced into the blind hole 220 until the elongate body transverse body 104B is adjacent to or contacts the glenoid 58. The elongate body 108D can be advanced into the blind hole 220 until a patient matched medial side of the glenoid baseplate 100D as indicated by the dash-dot line 99 in FIG. 1 contacts or nests with the natural or existing concavity of the glenoid 58. The elongate body 108D is located adjacent to or contacts the enclosed end 228.

Prior to advancing the elongate body 108D into the opening 224 the glenoid baseplate 100D can be rotationally oriented as indicated by the arrow 241 relative to the glenoid 58. For example, if there are rotational positioning indicia or features those features can be properly oriented by the surgeon. As noted above, the second side 124 of the transverse body 104B can be marked with “SUP” to indicate the portion of the transverse body 104B that should be oriented superiorly. In some embodiments the tooling interface 212 disposed on the coupling projection 210 of the transverse body 104B can be rotationally asymmetric such that a portion of the instrument that advances or holds the glenoid baseplate 100D can be easily visually confirmed to be properly oriented instead of or in addition to relying marking such as “SUP” for superior, “INF” for inferior, “ANT” for anterior, “POS” for posterior, etc.

FIG. 7C shows that following rotational positioning (if needed) and advancement of the glenoid baseplate 100D into the blind hole 220 the glenoid baseplate 100D can be secured to the scapula 55. One or more bone anchor 244 can be advanced through the transverse body 104B and into the bone. The bone anchor 244 can be directed through the transverse body 104B toward and/or into cortical bone of the scapula 55. In some techniques, the direction of the bone anchor 244 or the length thereof can be planned or selected prior to or during the surgery to assure a particular outcome. For instance the orientation or length can be planned or selected to assure the end of the bone anchor 244 opposite to the end coupled to the transverse body 104B is disposed in the cortical bone but not through the anterior surface 76 or the posterior surface 78 of the scapula 55. The orientation or length can be planned or selected to assure the end of the bone anchor 244 opposite to the end coupled to the transverse body 104B is disposed in the cortical bone and also extends through the anterior surface 76 or the posterior surface 78 of the scapula 55. FIG. 7C shows just one screw placed through the transverse body 104B into the scapula 55. Additional screws can be placed out of the section plane shown, e.g., superior to and/or inferior to the coupling projection 210. As discussed above, the glenoid baseplate 100D is configured such that two anchor apertures 132B are provided in superior and inferior positions and one anchor aperture 132B is provided at a posterior position. An anterior expanse of the transverse body 104B is provided in which the anchor aperture 132B is omitted. FIG. 7C shows that the glenoid baseplate 100D is secured without any bone anchor 244 between the position of the elongate body 108D and the anterior periphery of the transverse body 104B.

FIG. 7C shows that following the securing of the glenoid baseplate 100D to the scapula 55 the articular body 66 can be coupled with the glenoid baseplate 100D. The articular body 66 can be advanced an indicted by arrow 252 toward the coupling projection 210. The coupling projection 210 can be received in a corresponding tapered recess formed in the medial side of the articular body 66. The tapered recess and the coupling projection 210 can be configured to form a Morse taper connection. The Morse taper connection be sufficient to secure the articular body 66 to the glenoid baseplate 100D. In some embodiments, additional securement can be provided by a screw advanced through the articular body 66 into the threaded hole 180B of the coupling projection 210. The screw can engage threads in the threaded hole 180B as it is advanced from the open end 184B toward the enclosed end 188B. In some embodiments, a screw can provide the sole connection between the articular body 66 and the glenoid baseplate 100D by being advanced into the threaded hole 180B of the coupling projection 210, engaging threads in the threaded hole 180B as it is advanced from the open end 184B toward the enclosed end 188B.

### Terminology

As used herein, the relative terms “lateral” and “medial” shall be defined relative to the anatomy. Thus, medial refers to the direction toward the midline and lateral refers to the direction away from the midline.

Although certain embodiments and examples have been described herein, it will be understood by those skilled in the art that many aspects of the delivery systems shown and described in the present disclosure may be differently combined and/or modified to form still further embodiments or acceptable examples. All such modifications and variations are intended to be included herein within the scope of this disclosure. A wide variety of designs and approaches are possible. No feature, structure, or step disclosed herein is essential or indispensable.

For purposes of this disclosure, certain aspects, advantages, and novel features are described herein. It is to be understood that not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Moreover, while illustrative embodiments have been described herein, the scope of any and all embodiments having equivalent elements, modifications, omissions, combinations (e.g., of aspects across various embodiments), adaptations and/or alterations as would be appreciated by those in the art based on the present disclosure. The limitations in the claims are to be interpreted broadly based on the language employed in the claims and not limited to the examples described in the present specification or during the prosecution of the application, which examples are to be construed as non-exclusive. Further, the actions of the disclosed processes and methods may be modified in any manner, including by reordering actions and/or inserting additional actions and/or deleting actions. It is intended, therefore, that the specification and examples be considered as illustrative only, with a true scope and spirit being indicated by the claims and their full scope of equivalents.

Conditional language used herein, such as, among others, “can,” “might,” “may,” “e.g.,” and the like, unless specifically stated otherwise, or otherwise understood within the context as used, is generally intended to convey that some embodiments include, while other embodiments do not include, certain features, elements, and/or states. Thus, such conditional language is not generally intended to imply that features, elements, blocks, and/or states are in any way required for one or more embodiments or that one or more embodiments necessarily include logic for deciding, with or without author input or prompting, whether these features, elements and/or states are included or are to be performed in any particular embodiment.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±1%, ±5%, ±10%, ±15%, etc.). For example, “about 0.01 inches” includes “0.01 inches.” Phrases preceded by a term such as “substantially” include the recited phrase and should be interpreted based on the circumstances (e.g., as much as reasonably possible under the circumstances). For example, “substantially linear” includes “linear.”

1-18. (canceled)

19. A glenoid implant, comprising:
a baseplate, including:
a first body having a first side and a second side, the first side configured to face a scapula of a patient, and the second side disposed opposite from the first side, the first body having a periphery and a center;
a second body extending from the first side of the first body and defining a longitudinal axis, the longitudinal axis of the second body being parallel to an axis that extends through the center of the first body, the second body sized and configured to be at least partially received within a hole formed in the scapula of the patient,
wherein the center of the first body includes a feature for securing an articular component to the baseplate, and
wherein the first body defines a plurality of apertures, each aperture of the plurality of apertures disposed between the periphery of the first body and a periphery of the second body.

20. The glenoid implant of claim 19, wherein the feature for securing the articular component to the baseplate includes a threaded hole that extends from the first side of the first body towards the second body.

21. The glenoid implant of claim 19, wherein a first surface of the first body is separated from a second surface of the first body by a thickness.

22. The glenoid implant of claim 21, wherein the thickness of the first body varies between the periphery of the first body.

23. The glenoid implant of claim 22, wherein the periphery of the first body has a circular shape.

24. The glenoid implant of claim 22, wherein the second surface of the first body includes a patient specific surface.

25. The glenoid implant of claim 24, wherein the thickness of the first body varies according to a preoperative image of the scapula of the patient.

26. The glenoid implant of claim 20, wherein:
the second body includes a fastener body, and
the threaded hole extends into the fastener body.

27. The glenoid implant of claim 26, wherein the fastener body extends away from the first side of the first body and along a length of the second body.

28. The glenoid implant of claim 19, wherein the feature for securing the articular component to the baseplate includes a coupling body extending from the second side of the first body in a direction away from the first side of the first body.

29. The glenoid implant of claim 28, further comprising:
the articular component having an articular body, the articular body including a convex surface and a planar surface, and the articular body defining a recess that extends inwardly from the planar surface,
wherein the coupling body is tapered and is sized and configured to be received within the recess defined by the articular body.

30. The glenoid implant of claim 29, wherein the coupling body is symmetrical about an axis that extends through a center of the coupling body and the center of the first body.

31. The glenoid implant of claim 30, wherein a maximum outer diameter of the first body is greater than a maximum outer diameter of the coupling body.

32. The glenoid implant of claim 29, wherein an aspect ratio of the of the coupling body is greater than an aspect ratio of the first diameter.

33. The glenoid implant of claim 19, further comprising:
the articular component having an articular body, the articular body including a convex surface and a planar surface, and the articular body defining a recess that extends inwardly from the planar surface,
wherein the periphery of the first body tapers between the first side and the second side and is sized and configured to be received within the recess defined by the articular body.

34. The glenoid implant of claim 19, wherein the first body includes at least one tooling interface sized and configured to be engaged by a tool for use in implanting the baseplate.

35. A glenoid implant, comprising:
a baseplate, including:
a first body portion having a first surface, a second surface, a periphery, and a center, the first surface of the first body portion configured to face a scapula of a patient, the second surface of the first body separated from the first surface of the first body portion by a thickness,
a second body portion extending from the first surface, the second body portion sized and configured to be at least partially received within a hole formed in the scapula of the patient, wherein the second body portion includes a central longitudinal axis that is offset from a central axis defined by the first body portion; and
an articular component including an articular body, the articular body defining an aperture for coupling the articular component to the baseplate,
wherein the center of the first body portion of the baseplate includes a feature for securing the articular component to the baseplate.

36. The glenoid implant of claim 35, wherein:
the feature for securing the articular component includes a threaded hole that extends inwardly from the first surface of the first body portion towards the second surface of the first body portion;
the periphery of the first body portions tapers between the first surface and the second surface and is sized and configured to be at least partially received within the aperture defined by the articular body.

37. The glenoid implant of claim 35, wherein:
the feature for securing the articular component to the baseplate includes a coupling body extending from the second side of the first body portion away from the first surface of the first body portion; and
the coupling body is tapered and is sized and configured to be received within the aperture defined by the articular body.

38. The glenoid implant of claim 35, wherein the thickness of the first body portion of the baseplate varies along a width of the first body portion such that the baseplate includes an augment portion.
